(19) United States
(12) Patent Application Publication
(10) Pub. No.: US 2021/0024390 A1
(43) Pub. Date: Jan. 28, 2021

(54) METHOD FOR ENHANCING NITROGEN REMOVAL BY DENITRIFICATION IN HORIZONTAL SUBSURFACE-FLOW CONSTRUCTED WETLAND

(71) Applicant: Nanjing Institute of Geography and Limnology, Chinese Academy of Sciences, Nanjing (CN)

(72) Inventors: Fan KE, Nanjing (CN); Fei DIAO, Nanjing (CN); Wenchao LI, Nanjing (CN); Jizheng PAN, Nanjing (CN)

(73) Assignee: Nanjing Institute of Geography and Limnology, Chinese Academy of Sciences, Nanjing (CN)

(21) Appl. No.: 16/901,094

(22) Filed: Jun. 15, 2020

(30) Foreign Application Priority Data
Jul. 23, 2019 (CN) 201910666462.X

(51) Int. Cl. C02F 3/12; C12Q 1/02; C12N 1/20; C02F 3/34

(52) U.S. Cl. CPC C02F 3/1205; C12Q 1/02; C12N 1/20; C02F 2101/16; C02F 2209/06; C02F 2305/06; C02F 2209/02; C02F 3/34

(57) ABSTRACT

A method for enhancing nitrogen removal by denitrification in a horizontal subsurface-flow constructed wetland, which includes three treatment steps of strain acclimation, strain screening and strain pouring. Firstly, the enrichment of aerobic denitrifying bacteria is achieved through acclimation culture, and the denitrification advantages of the strains are improved to obtain acclimated strains; meanwhile in the present technical solution, after obtained, the acclimated strains are screened for multiple times to remove aging strains and some strains incapable of performing denitrification from sludge and obtain strains truly having good denitrification effects as strains to be poured; and finally the strains to be poured as obtained by the screening is combined with a biocarrier to obtain an immobilized filler and thus prevent the strains from loss.

### CROSS REFERENCE TO RELATED APPLICATIONS

The present application claims priority to Chinese Patent Application No. 201910666462.X (filed on Jul. 23, 2019), the entire content of which is incorporated herein by reference in its complete entirety.

### TECHNICAL FIELD

The present invention relates to the technical field of wetland treatment, and in particular to a method for enhancing nitrogen removal by denitrification in a horizontal subsurface-flow constructed wetland.

### BACKGROUND

In the process of wetland management and operation, the sewage needs to pass through a micro-aerated vertical-flow wetland and then enter a horizontal subsurface-flow constructed wetland. During this process, the sewage has high dissolved oxygen, so that it is in an aerobic state in the horizontal subsurface-flow constructed wetland. However, the traditional theory believes that denitrification is a strict anaerobic process. Oxygen inhibits denitrifying enzymes and is the preferred electron acceptor during the oxidation of an organic matter, thereby preventing nitrate nitrogen and nitrite nitrogen from being electron acceptors.

In recent years, researchers have gradually discovered the existence of aerobic denitrifying bacteria. People have isolated some aerobic denitrifying bacteria in various environments such as soil, ditches, ponds, activated sludge, sediments, etc. However, the aerobic denitrification bacteria cannot effectively complete the process of nitrogen removal by denitrification in the horizontal subsurface-flow constructed wetland due to the small number and unobvious advantages thereof.

In view of the above situation, we provide a method for enhancing nitrogen removal by denitrification in a horizontal subsurface-flow constructed wetland, which needs to improve the denitrification performance of the aerobic denitrifying bacteria. This is one of the problems we need to solve urgently.

### SUMMARY

An objective of the present invention is to provide a method for enhancing nitrogen removal by denitrification in a horizontal subsurface-flow constructed wetland, so as to solve the problem of the prior art.

To achieve the above objectives, the present invention provides the following technical solution.

A method for enhancing nitrogen removal by denitrification in a horizontal subsurface-flow constructed wetland, includes the following steps:

1) conducting strain acclimation;

2) conducting strain screening;

3) conducting strain pouring; and

4) ending the operation.

The present technical solution is used for the horizontal subsurface-flow constructed wetland. In recent years, researchers have gradually discovered the existence of aerobic denitrifying bacteria. People have isolated some aerobic denitrifying bacteria in various environments such as soil, ditches, ponds, activated sludge, sediments, etc. However, due to the small number and unobvious advantages of the aerobic denitrification bacteria, the present technical solution provides a method for enhancing nitrogen removal by denitrification in the horizontal subsurface-flow constructed wetland.

More preferably, the method includes the following steps:

1) conducting strain acclimation:

A) preparing a carbon source, a nitrogen source, activated sludge obtained after enrichment culture and a culture solution, and concentrating the activated sludge to obtain a concentrated sludge solution for later use;

B) pouring the culture solution prepared in step A into a container, then adding the concentrated sludge solution, adjusting the pH, and then adding the carbon source and the nitrogen source, so that in the container the carbon content is 390-410 ppm, the nitrogen content is about 18-22 ppm, and the ratio of carbon to nitrogen is C/N=20;

C) aerating by a small air pump so that the dissolved oxygen in the container is DO≥5, and conducting culture at room temperature for 6-7 consecutive days to obtain acclimated strains;

2) conducting strain screening:

a) weighing raw materials in proportion to formulate a screening medium, adjusting the pH to 7-7.2, sterilizing at 120-125° C. for 20-30 min, and cooling for later use;

b) transferring a mixed muddy water liquor from the container after acclimation of step 1), conducting gradient dilution on the mixed muddy water liquor, coating the diluted solution onto the screening medium, culturing for 2-3 days, picking colonies with blue halos around the medium after the colonies are formed, and repeatedly streaking to obtain primarily-screened bacteria;

c) inoculating the primarily-screened bacteria in an Erlenmeyer flask filled with a liquid culture medium, culturing and sampling to determine the removal rates of ammonia nitrogen and total nitrogen, and taking strains whose determined removal levels of ammonia nitrogen and total nitrogen reach more than 85% of the normal removal levels of the strains, as secondarily-screened bacteria;

3) conducting strain pouring:

a) inoculating the secondarily-screened bacteria in a TSA medium, culturing them in a shake flask at 150-160 r/min, and finishing the culture to obtain a bacterial suspension when the secondarily-screened bacteria are in a logarithmic growth phase according to a growth curve of the secondarily-screened bacteria;

b) taking and putting the bacterial suspension prepared in step a) into an Erlenmeyer flask, then putting a treated biocarrier into the Erlenmeyer flask, culturing in a shaker for 6-8 h for primary biofilm formation, replacing a fresh medium after the primary biofilm formation, and culturing in the shaker by using the late logarithmic growth phase of the secondarily-screened bacteria as a cycle, so as to complete immobilization and obtain a immobilized filler;

c) controlling the environmental temperature and pH of the wetland, and pouring the immobilized filler treated in step b) into the wetland to complete the nitrogen removal by denitrification;

4) ending the operation.

More preferably, the method includes the following steps:

1) conducting strain acclimation:

A. preparing a carbon source, a nitrogen source, activated sludge obtained after enrichment culture and a culture solution, and concentrating the activated sludge to obtain a concentrated sludge solution for later use;

B. pouring the culture solution prepared in step A into a container with a volume of 15 L, then adding the concentrated sludge solution, adjusting the pH to 6-8, and then adding the carbon source and the nitrogen source, so that in the container the carbon content is 390-410 ppm, the nitrogen content is about 18-22 ppm, and the ratio of carbon to nitrogen is C/N=20;

C. aerating by a small air pump so that the dissolved oxygen content in the container is DO≥5, and conducting culture at room temperature for 6-7 consecutive days to obtain acclimated strains, where the carbon source and the nitrogen source are added once every 24 h, and the addition amount of the carbon source and the nitrogen source is the same as that in step B; aeration culture in step 1) is used for enriching the aerobic denitrifying strains, while improving the denitrification performance of the aerobic denitrifying strains, which is convenient for subsequent strain screening and denitrification treatment operations in the wetland;

2) conducting strain screening:

a) weighing raw materials in proportion to formulate a screening medium, adjusting the pH to 7-7.2, sterilizing at 120-125° C. for 20-30 min, and cooling for later use;

b) transferring a mixed muddy water liquor from the container after acclimation of step 1), conducting gradient dilution on the mixed muddy water liquor, coating the diluted solution onto the screening medium, culturing at 30-35° C. for 2-3 days, picking colonies with blue halos around the medium after the colonies are formed, and repeatedly streaking to obtain primarily-screened bacteria, where in step b), primary screening is conducted through the screening medium, since the screening medium contains bromothymol blue as an indicator, when the pH is greater than 7.6, the medium will turn blue, and the strain denitrification is a process of alkali generation, denitrification in the screening medium will cause the pH of the medium to increase and thus result in occurrence of the blue halos around the colonies, and the primary screening of the aerobic denitrifying strains is completed through this technical solution, thereby removing aging and dead strains and strains incapable of performing denitrification from the strains;

c) inoculating the primarily-screened bacteria in an Erlenmeyer flask filled with a liquid culture medium, culturing at a constant temperature of 30-35° C. by shaking for 24-48 h, sampling to determine the removal rates of ammonia nitrogen and total nitrogen, and taking strains whose determined removal levels of ammonia nitrogen and total nitrogen reach more than 85% of the normal removal levels of the strains, as secondarily-screened bacteria, where for the secondary screening conducted in step c), because certain strains also generate alkali during the metabolic process, the pH of the culture medium is increased, so that in this step by the secondary screening, it is further determined that the resultant strains have the aerobic denitrification performance, and the doped strains incapable of performing aerobic denitrification are removed; and through the determined removal levels of ammonia nitrogen and total nitrogen, the strains having the excellent aerobic denitrification performance are screened out as the strains to be poured, that is, i.e., the secondarily-screened bacteria;

3) conducting strain pouring; and

a) inoculating the secondarily-screened bacteria in a TSA medium, culturing them in a shake flask at 150-160 r/min, and finishing the culture to obtain a bacterial suspension when the secondarily-screened bacteria are in a logarithmic growth phase according to a growth curve of the secondarily-screened bacteria;

b) taking and putting the bacterial suspension prepared in step a) into an Erlenmeyer flask, then putting a treated biocarrier into the Erlenmeyer flask, culturing in a shaker under culture conditions of 30-32° C. and 135-145 r/min for 6-8 h for primary biofilm formation, replacing a fresh medium after the primary biofilm formation, and culturing in the shaker by using the late logarithmic growth phase of the secondarily-screened bacteria as a cycle, so as to complete immobilization and obtain an immobilized filler, where for the strain pouring in step 3), firstly the bacterial suspension of the secondarily-screened bacteria is prepared, and then the bacterial suspension is combined with a biocarrier to complete the immobilization of the strains and prepare the immobilized filler;

c) controlling the environmental temperature of the wetland at 5-7° C. and the pH of the wetland at 6.5-7.5, and pouring the immobilized filler treated in step b) into the wetland to complete the nitrogen removal by denitrification, where in step c) the environment of the wetland is regulated, so that the aerobic denitrifying strains conduct the denitrification process rapidly and effectively in this environment;

4) ending the operation.

More preferably, in step 1), in one cycle, the air-pump aeration time is 23.5 h, and the time for settling and pouring of the carbon source and the nitrogen source is 0.5 h.

More preferably, in step 1), the carbon source is one of methanol, ethanol, sodium acetate, polycaprolactone (PCL), and a nutshell.

More preferably, the nutshell is one of a coconut shell, a walnut shell, and polycaprolactone (PCL).

In the present technical solution, one of methanol, ethanol, sodium acetate, polycaprolactone (PCL) and the nutshell is selected as the carbon source, and the more preferred technical solutions are the coconut shell and the walnut shell, which can completely replace ethanol; and the raw materials are easy to get and the wastes are utilized, so that the present technical solution is suitable for large-scale use and has a low cost.

More preferably, in step b) of step 3), the biocarrier is one of sodium alginate, a mycelium pellet, and a polyurethane foam.

In the present technical solution, the biocarrier is selected from one of sodium alginate, the mycelium pellet, and the polyurethane foam. The polyurethane foam can immobilize the aerobic denitrifying strains in the inner and outer layers of the carrier through organic adsorption, and has a large absorptive capacity and strong anti-impact and anti-load capacities. The sodium alginate can embed the aerobic denitrifying strains in the carrier. The most preferred one of the three raw materials is the mycelium pellet, which not only has a low cost and is easy to cultivate, but also has good biocompatibility and can enable the aerobic denitrifying strains to effectively maintain the biological activity and have a good adsorption performance.

More preferably, in step a) of step 2), the concentration ratio of various substances in the screening medium is as follows: 20 g/L of agar, 1 g/L of KNO3, 1 g/L of KH2PO4, 0.5 g/L of FeCl2.6H2O, 0.2 g/L of CaCl2.7H2O, 1 g/L of MgSO4.7H2O, 8.5 g/L of sodium succinate, and 1 mL/L of bromothymol blue (BTB).

More preferably, in step c) of step 2), the concentration ratio of various substances in the liquid medium is as follows: 1 g/L of KNO3, 1 g/L of KH2PO4, 0.05 g/L of FeCl2.6H2O, 0.02 g/L of CaCl2.7H2O, 1 g/L of MgSO4.7H2O, and 8.5 g/L of sodium succinate, and the liquid medium is sterilized at 120° C. for 20-25 min, and cooled for later use.

In the present technical solution, the effluent of the sewage treatment plant in Chengjiang County is selected as the culture medium.

Compared with the prior art, the present invention has the following beneficial effects.

The present invention includes three treatment steps of strain acclimation, strain screening and strain pouring. There are few aerobic denitrifying bacteria in the nature since the aerobic denitrifying bacteria are not predominance strains in the environment. Up to now, people have isolated some aerobic denitrifying bacteria in various environments such as soil, ditches, ponds, activated sludge, sediments, etc. However, due to the small number and unobvious advantages of the aerobic denitrification bacteria, in the present technical solution, firstly the enrichment of aerobic denitrifying bacteria is achieved through acclimation culture, and the denitrification advantages of the strains are improved to obtain acclimated strains; meanwhile in the present technical solution, after obtained, the acclimated strains are screened for multiple times to remove aging strains and some strains incapable of performing denitrification from sludge and obtain strains truly having good denitrification effects as strains to be poured; and finally the strains to be poured as obtained by the screening is combined with a biocarrier to obtain an immobilized filler, which can not only improve the biochemical reaction rate of the aerobic denitrifying strains, but also protect the strains and prevent the strains from loss.

The present invention designs a method for enhancing nitrogen removal by denitrification in a horizontal subsurface-flow constructed wetland. The design of steps is reasonable and the process operation is simple, which not only effectively achieves the aerobic denitrification operation in the horizontal subsurface-flow constructed wetland, but also ensures the biological activity and denitrification performance of the aerobic denitrifying strains and enhances the denitrification of the horizontal subsurface-flow constructed wetland. For the method, the cost of the whole process is low, the raw materials are easy to get, and the method has high practicability and wide application prospects.

### DETAILED DESCRIPTION

The following clearly and completely describes the technical solutions in the examples of the present invention. Apparently, the described examples are merely some examples of the present invention rather than all of the examples. All other examples obtained by a person of ordinary skill in the art based on the examples of the present invention without creative efforts shall fall within the protection scope of the present invention.

Experiments of nitrogen removal by denitrification in a horizontal subsurface-flow constructed wetland are conducted through the following examples 1-3, and specifically include the following steps.

In the experiments, the concentration ratio of various substances in the liquid medium is as follows: 1 g/L of KNO3, 1 g/L of KH2PO4, 0.05 g/L of FeCl2.6H2O, 0.02 g/L of CaCl2.7H2O, 1 g/L of MgSO4.7H2O, and 8.5 g/L of sodium succinate, and the liquid medium is sterilized at 120° C. for 20-25 min, and cooled for later use.

The concentration ratio of various substances in the screening medium is as follows: 20 g/L of agar, 1 g/L of KNO3, 1 g/L of KH2PO4, 0.5 g/L of FeCl2.6H2O, 0.2 g/L of CaCl2.7H2O, 1 g/L of MgSO4.7H2O, 8.5 g/L of sodium succinate, and 1 mL/L of bromothymol blue (BTB).

### EXAMPLE 1

Step 1: strain acclimation: firstly a carbon source, a nitrogen source, activated sludge obtained after enrichment culture and a culture solution were prepared, and the activated sludge was concentrated to obtain a concentrated sludge solution for later use. Then the culture solution prepared was poured into a 15 L container, and then added with the concentrated sludge solution, the pH of the mixture was adjusted to 6, and then the mixture was added with the carbon source and the nitrogen source, such that in the container the carbon content was 390 ppm and the nitrogen content was about 19.5 ppm, and the carbon/nitrogen ratio was C/N=20. Finally, aeration was conducted by a small air pump so that the dissolved oxygen in the container was DO≥5, culture was conducted at room temperature, and added with the carbon source and the nitrogen source once every 24 h, where the addition amount of the carbon source and the nitrogen source was the same as that in step B, and in one cycle the air-pump aeration time was 23.5 h, and the time of settling and pouring of the carbon source and the nitrogen source was 0.5 h. The culture was conducted for 6 consecutive days to obtain acclimated strains, where the carbon source was polycaprolactone (PCL), and the nitrogen source was potassium nitrate.

Then strain screening was conducted. Raw materials were weighed in proportion to formulate a screening medium, the pH of the screening medium was adjusted to 7, and the screening medium was sterilized at 120° C. for 20 min and then cooled for later use. A mixed muddy water liquor was transferred from the container after acclimation, and subjected to gradient dilution. The diluted solution was coated onto the screening medium, and cultured at 30° C. for 2 days. After the colonies were formed, the colonies with blue halos around the medium were picked, and repeatedly streaked to obtain primarily-screened bacteria. The primarily-screened bacteria was inoculated in an Erlenmeyer flask filled with a liquid culture medium, cultured at a constant temperature of 30° C. by shaking for 24 h, and sampled to determine the removal rates of ammonia nitrogen and total nitrogen. The strains whose determined removal levels of ammonia nitrogen and total nitrogen reach more than 85% of the normal removal levels of the strains, were taken as secondarily-screened bacteria.

Then strain pouring was conducted. The secondarily-screened bacteria were inoculated in a TSA medium, and cultured in a shaker at 150 r/min. The culture was finished to obtain a bacterial suspension when the secondarily-screened bacteria were in a logarithmic growth phase according to a growth curve of the secondarily-screened bacteria. The prepared bacterial suspension was taken and put into an Erlenmeyer flask, and then added with a treated biocarrier. Culture was conducted in a shaker under culture conditions of 30° C. and 135 r/min for 6 h for primary biofilm formation. A fresh medium was replaced after the primary biofilm formation, and cultured in the shaker by using the late logarithmic growth phase of the secondarily-screened bacteria as a cycle, so as to complete immobilization and obtain a immobilized filler. Then the environmental temperature of the wetland was controlled at 5° C. and the pH of the wetland was controlled at 6.5, and the immobilized filler was poured into the wetland to complete the nitrogen removal by denitrification, where the biocarrier was sodium alginate.

### EXAMPLE 2

Step 1: strain acclimation: firstly a carbon source, a nitrogen source, activated sludge obtained after enrichment culture and a culture solution were prepared, and the activated sludge was concentrated to obtain a concentrated sludge solution for later use. Then the culture solution prepared in step A was poured into a 15 L container, and then added with the concentrated sludge solution, the pH of the mixture was adjusted to 7, and then the mixture was added with the carbon source and the nitrogen source, such that in the container the carbon content was 400 ppm and the nitrogen content was about 20 ppm, and the carbon/nitrogen ratio was C/N=20. Finally, aeration was conducted by a small air pump so that the dissolved oxygen in the container was DO≥5, culture was conducted at room temperature, and added with the carbon source and the nitrogen source once every 24 h, where the addition amount of the carbon source and the nitrogen source was the same as that in step B, and in one cycle the air-pump aeration time was 23.5 h, and the time of settling and pouring of the carbon source and the nitrogen source was 0.5 h. The culture was conducted for 6 consecutive days to obtain acclimated strains, where the carbon source was the coconut shell, and the nitrogen source was potassium nitrate.

Then strain screening was conducted. Raw materials were weighed in proportion to formulate a screening medium, the pH of the screening medium was adjusted to 7.1, and the screening medium was sterilized at 123° C. for 25 min and then cooled for later use. A mixed muddy water liquor was transferred from the container after acclimation, and subjected to gradient dilution. The diluted solution was coated onto the screening medium, and cultured at 33° C. for 2 days. After the colonies were formed, the colonies with blue halos around the medium were picked, and repeatedly streaked to obtain primarily-screened bacteria. The primarily-screened bacteria was inoculated in an Erlenmeyer flask filled with a liquid culture medium, cultured at a constant temperature of 33° C. by shaking for 36 h, and sampled to determine the removal rates of ammonia nitrogen and total nitrogen. The strains whose determined removal levels of ammonia nitrogen and total nitrogen reach more than 85% of the normal removal levels of the strains, were taken as secondarily-screened bacteria.

Then strain pouring was conducted. The secondarily-screened bacteria were inoculated in a TSA medium, and cultured in a shaker at 155 r/min. The culture was finished to obtain a bacterial suspension when the secondarily-screened bacteria were in a logarithmic growth phase according to a growth curve of the secondarily-screened bacteria. The prepared bacterial suspension was taken and put into an Erlenmeyer flask, and then added with a treated biocarrier. Culture was conducted in a shaker under culture conditions of 31° C. and 140 r/min for 7 h for primary biofilm formation. A fresh medium was replaced after the primary biofilm formation, and cultured in the shaker by using the late logarithmic growth phase of the secondarily-screened bacteria as a cycle, so as to complete immobilization and obtain a immobilized filler. Then the environmental temperature of the wetland was controlled at 6° C. and the pH of the wetland was controlled at 7, and the immobilized filler was poured into the wetland to complete the nitrogen removal by denitrification, where the biocarrier was sodium alginate.

### EXAMPLE 3

Step 1: strain acclimation: firstly a carbon source, a nitrogen source, activated sludge obtained after enrichment culture and a culture solution were prepared, and the activated sludge was concentrated to obtain a concentrated sludge solution for later use. Then the culture solution prepared in step A was poured into a 15 L container, and then added with the concentrated sludge solution, the pH of the mixture was adjusted to 7, and then the mixture was added with the carbon source and the nitrogen source, such that in the container the carbon content was 410 ppm and the nitrogen content was about 20.5 ppm, and the carbon/nitrogen ratio was C/N=20. Finally, aeration was conducted by a small air pump so that the dissolved oxygen in the container was DO≥5, culture was conducted at room temperature, and added with the carbon source and the nitrogen source once every 24 h, where the addition amount of the carbon source and the nitrogen source was the same as that in step B, and in one cycle the air-pump aeration time was 23.5 h, and the time of settling and pouring of the carbon source and the nitrogen source was 0.5 h. The culture was conducted for 6-7 consecutive days to obtain acclimated strains, where the carbon source was the walnut shell, and the nitrogen source was potassium nitrate.

Then strain screening was conducted. Raw materials were weighed in proportion to formulate a screening medium, the pH of the screening medium was adjusted to 7.2, and the screening medium was sterilized at 125° C. for 20-30 min and then cooled for later use. A mixed muddy water liquor was transferred from the container after acclimation, and subjected to gradient dilution. The diluted solution was coated onto the screening medium, and cultured at 30-35° C. for 3 days. After the colonies were formed, the colonies with blue halos around the medium were picked, and repeatedly streaked to obtain primarily-screened bacteria. The primarily-screened bacteria was inoculated in an Erlenmeyer flask filled with a liquid culture medium, cultured at a constant temperature of 30-35° C. by shaking for 48 h, and sampled to determine the removal rates of ammonia nitrogen and total nitrogen. The strains whose determined removal levels of ammonia nitrogen and total nitrogen reach more than 85% of the normal removal levels of the strains, were taken as secondarily-screened bacteria.

Then strain pouring was conducted. The secondarily-screened bacteria were inoculated in a TSA medium, and cultured in a shaker at 160 r/min. The culture was finished to obtain a bacterial suspension when the secondarily-screened bacteria were in a logarithmic growth phase according to a growth curve of the secondarily-screened bacteria. The prepared bacterial suspension was taken and put into an Erlenmeyer flask, and then added with a treated biocarrier. Culture was conducted in a shaker under culture conditions of 32° C. and 145 r/min for 8 h for primary biofilm formation. A fresh medium was replaced after the primary biofilm formation, and cultured in the shaker by using the late logarithmic growth phase of the secondarily-screened bacteria as a cycle, so as to complete immobilization and obtain a immobilized filler. Then the environmental temperature of the wetland was controlled at 7° C. and the pH of the wetland was controlled at 7.5, and the immobilized filler was poured into the wetland to complete the nitrogen removal by denitrification, where the biocarrier was sodium alginate.

### Comparative Example 1

Step 1: strain acclimation: firstly a carbon source, a nitrogen source, activated sludge obtained after enrichment culture and a culture solution were prepared, and the activated sludge was concentrated to obtain a concentrated sludge solution for later use. Then the culture solution prepared in step A was poured into a 15 L container, and then added with the concentrated sludge solution, the pH of the mixture was adjusted to 7, and then the mixture was added with the carbon source and the nitrogen source, such that in the container the carbon content was 400 ppm and the nitrogen content was about 20 ppm, and the carbon/nitrogen ratio was C/N=20. Finally, aeration was conducted by a small air pump so that the dissolved oxygen in the container was DO≥5, culture was conducted at room temperature, and added with the carbon source and the nitrogen source once every 24 h, where the addition amount of the carbon source and the nitrogen source was the same as that in step B, and in one cycle the air-pump aeration time was 23.5 h, and the time of settling and pouring of the carbon source and the nitrogen source was 0.5 h. The culture was conducted for 6 consecutive days to obtain acclimated strains, where the carbon source was the coconut shell, and the nitrogen source was potassium nitrate.

Then strain pouring was conducted. The secondarily-screened bacteria were inoculated in a TSA medium, and cultured in a shaker at 155 r/min. The culture was finished to obtain a bacterial suspension when the secondarily-screened bacteria were in a logarithmic growth phase according to a growth curve of the secondarily-screened bacteria. The prepared bacterial suspension was taken and put into an Erlenmeyer flask, and then added with a treated biocarrier. Culture was conducted in a shaker under culture conditions of 31° C. and 140 r/min for 7 h for primary biofilm formation. A fresh medium was replaced after the primary biofilm formation, and cultured in the shaker by using the late logarithmic growth phase of the secondarily-screened bacteria as a cycle, so as to complete immobilization and obtain a immobilized filler. Then the environmental temperature of the wetland was controlled at 6° C. and the pH of the wetland was controlled at 7, and the immobilized filler was poured into the wetland to complete the nitrogen removal by denitrification, where the biocarrier was sodium alginate.

### Comparative Example 2

Step 1: strain acclimation: firstly a carbon source, a nitrogen source, activated sludge obtained after enrichment culture and a culture solution were prepared, and the activated sludge was concentrated to obtain a concentrated sludge solution for later use. Then the culture solution prepared in step A was poured into a 15 L container, and then added with the concentrated sludge solution, the pH of the mixture was adjusted to 7, and then the mixture was added with the carbon source and the nitrogen source, such that in the container the carbon content was 400 ppm and the nitrogen content was about 20 ppm, and the carbon/nitrogen ratio was C/N=20. Finally, aeration was conducted by a small air pump so that the dissolved oxygen in the container was DO≥5, culture was conducted at room temperature, and added with the carbon source and the nitrogen source once every 24 h, where the addition amount of the carbon source and the nitrogen source was the same as that in step B, and in one cycle the air-pump aeration time was 23.5 h, and the time of settling and pouring of the carbon source and the nitrogen source was 0.5 h. The culture was conducted for 6 consecutive days to obtain acclimated strains, where the carbon source was the coconut shell, and the nitrogen source was potassium nitrate.

Then strain screening was conducted. Raw materials were weighed in proportion to formulate a screening medium, the pH of the screening medium was adjusted to 7.1, and the screening medium was sterilized at 123° C. for 25 min and then cooled for later use. A mixed muddy water liquor was transferred from the container after acclimation, and subjected to gradient dilution. The diluted solution was coated onto the screening medium, and cultured at 33° C. for 2 days. After the colonies were formed, the colonies with blue halos around the medium were picked, and repeatedly streaked to obtain primarily-screened bacteria. The primarily-screened bacteria was inoculated in an Erlenmeyer flask filled with a liquid culture medium, cultured at a constant temperature of 33° C. by shaking for 36 h, and sampled to determine the removal rates of ammonia nitrogen and total nitrogen. The strains whose determined removal levels of ammonia nitrogen and total nitrogen reach more than 85% of the normal removal levels of the strains, were taken as secondarily-screened bacteria.

Then strain pouring was conducted. The environmental temperature of the wetland was controlled at 6° C. and the pH of the wetland was controlled at 7, and the immobilized filler was poured into the wetland to complete the nitrogen removal by denitrification.

Conclusion: by comparing the effects of nitrogen removal by denitrification of strains of Examples 1-3, Comparative Example 1, and Comparative Example 3, the following conclusions can be obtained:

1. in the present technical solution, acclimated strains are obtained through acclimation culture, the acclimated strains have better denitrification advantages, and meanwhile the process of acclimation culture also achieves enrichment of the aerobic denitrifying strains;

2. by comparing Examples 1-3 with Comparative Example 1, it can be found that in the present technical solution by multiple screening of the acclimated strains, the aging strains and some strains incapable of performing denitrification can be removed from the sludge, and the strains truly having good denitrification effects can be obtained as the strains to be poured;

3. by comparing Examples 1-3 and Comparative Example 2, it can be found that in the present technical solution by combining the screened strains to be poured with a biocarrier, the immobilized filler is obtained, which can not only improve the biochemical reaction rate of the aerobic denitrifying strains, but also protect the strains and prevent the strains from loss.

The present invention designs a method for enhancing nitrogen removal by denitrification in a horizontal subsurface-flow constructed wetland. The design of steps is reasonable and the process operation is simple, which not only effectively achieves the aerobic denitrification operation in the horizontal subsurface-flow constructed wetland, but also ensures the biological activity and denitrification performance of the aerobic denitrifying strains and enhances the denitrification of the horizontal subsurface-flow constructed wetland. For the method, the cost of the whole process is low, the raw materials are easy to get, and the method has high practicability and wide application prospects.

It apparent for those skilled in the art that the present invention is not limited to the details of the above exemplary examples, and that the present invention may be implemented in other specific forms without departing from the spirit or basic features of the present invention. The examples should be regarded as exemplary and non-limiting in every respect, and the scope of the present invention is defined by the appended claims rather than the above description. Therefore, all changes falling within the meaning and scope of equivalent elements of the claims should be included in the present invention.

What is claimed is:

1. A method for enhancing nitrogen removal by denitrification in a horizontal subsurface-flow constructed wetland, comprising the following steps:
1) conducting strain acclimation;
2) conducting strain screening;
3) conducting strain pouring; and
4) ending the operation.

2. The method of claim 1, wherein conducting strain acclimation comprises:
A. preparing a carbon source, a nitrogen source, activated sludge obtained after enrichment culture and a culture solution, and concentrating the activated sludge to obtain a concentrated sludge solution for later use;
B. pouring the culture solution prepared in step A into a container, then adding the concentrated sludge solution, adjusting the pH, and then adding the carbon source and the nitrogen source, so that in the container the carbon content is 390-410 ppm, the nitrogen content is about 18-22 ppm, and a ratio of carbon to nitrogen is C/N=20; and
C. aerating by a small air pump so that the dissolved oxygen in the container is DO≥5, and conducting culture at room temperature for 6-7 consecutive days to obtain acclimated strains;

[symbol]

3. The method of claim 2, wherein conducting strain screening comprises:
a) weighing raw materials in proportion to formulate a screening medium, adjusting the pH to 7-7.2, sterilizing at 120-125° C. for 20-30 min, and cooling for later use;
b) transferring a mixed muddy water liquor from the container after acclimation of step 1), conducting gradient dilution on the mixed muddy water liquor, coating the diluted solution onto the screening medium, culturing for 2-3 days, picking colonies with blue halos around the medium after the colonies are formed, and repeatedly streaking to obtain primarily-screened bacteria; and
c) inoculating the primarily-screened bacteria in an Erlenmeyer flask filled with a liquid culture medium, culturing and sampling to determine the removal rates of ammonia nitrogen and total nitrogen, and taking strains whose determined removal levels of ammonia nitrogen and total nitrogen reach more than 85% of the normal removal levels of the strains, as secondarily-screened bacteria.

4. The method of claim 3, wherein conducting strain pouring comprises:
a) inoculating the secondarily-screened bacteria in a TSA medium, culturing them in a shake flask at 150-160 r/min, and finishing the culture to obtain a bacterial suspension when the secondarily-screened bacteria are in a logarithmic growth phase according to a growth curve of the secondarily-screened bacteria;
b) taking and putting the bacterial suspension prepared in step a) into an Erlenmeyer flask, then putting a treated biocarrier into the Erlenmeyer flask, culturing in a shaker for 6-8 h for primary biofilm formation, replacing a fresh medium after the primary biofilm formation, and culturing in the shaker by using the late logarithmic growth phase of the secondarily-screened bacteria as a cycle, so as to complete immobilization and obtain a immobilized filler; and
c) controlling the environmental temperature and pH of the wetland, and pouring the immobilized filler treated in step b) into the wetland to complete the nitrogen removal by denitrification;

[symbol]

5. The method of claim 1, wherein conducting strain acclimation comprises:
A. preparing a carbon source, a nitrogen source, activated sludge obtained after enrichment culture and a culture solution, and concentrating the activated sludge to obtain a concentrated sludge solution for later use;
B. pouring the culture solution prepared in step A into a container with a volume of 15 L, then adding the concentrated sludge solution, adjusting the pH to 6-8, and then adding the carbon source and the nitrogen source, so that in the container the carbon content is 390-410 ppm, the nitrogen content is about 18-22 ppm, and a ratio of carbon to nitrogen is C/N=20; and
C. aerating by a small air pump so that the dissolved oxygen content in the container is DO≥5, and conducting culture at room temperature for 6-7 consecutive days to obtain acclimated strains, wherein the carbon source and the nitrogen source are added once every 24 h, and the addition amount of the carbon source and the nitrogen source is the same as that in step B.

6. The method of claim 5, wherein conducting strain screening comprises:
a) weighing raw materials in proportion to formulate a screening medium, adjusting the pH to 7-7.2, sterilizing at 120-125° C. for 20-30 min, and cooling for later use;
b) transferring a mixed muddy water liquor from the container after acclimation of step 1), conducting gradient dilution on the mixed muddy water liquor, coating the diluted solution onto the screening medium, culturing at 30-35° C. for 2-3 days, picking colonies with blue halos around the medium after the colonies are formed, and repeatedly streaking to obtain primarily-screened bacteria; and
c) inoculating the primarily-screened bacteria in an Erlenmeyer flask filled with a liquid culture medium, culturing at a constant temperature of 30-35° C. by shaking for 24-48 h, sampling to determine the removal rates of ammonia nitrogen and total nitrogen, and taking strains whose determined removal levels of ammonia nitrogen and total nitrogen reach more than 85% of the normal removal levels of the strains, as secondarily-screened bacteria.

7. The method of claim 6, wherein conducting strain pouring comprises:
a) inoculating the secondarily-screened bacteria in a TSA medium, culturing them in a shake flask at 150-160 r/min, and finishing the culture to obtain a bacterial suspension when the secondarily-screened bacteria are in a logarithmic growth phase according to a growth curve of the secondarily-screened bacteria;
b) taking and putting the bacterial suspension prepared in step a) into an Erlenmeyer flask, then putting a treated biocarrier into the Erlenmeyer flask, culturing in a shaker under culture conditions of 30-32° C. and 135-145 r/min for 6-8 h for primary biofilm formation, replacing a fresh medium after the primary biofilm formation, and culturing in the shaker by using the late logarithmic growth phase of the secondarily-screened bacteria as a cycle, so as to complete immobilization and obtain a immobilized filler; and
c) controlling the environmental temperature of the wetland at 5-7° C. and the pH of the wetland at 6.5-7.5, and pouring the immobilized filler treated in step b) into the wetland to complete the nitrogen removal by denitrification.

8. The method of claim 7, wherein in the conducting strain acclimation:
in one cycle, an air-pump aeration time is 23.5 h, and
a time for settling and pouring of the carbon source and the nitrogen source is 0.5 h.

9. The method of claim 7, wherein in the conducting strain acclimation:
the carbon source is one of methanol, ethanol, sodium acetate, polycaprolactone (PCL), and a nutshell, and
the nitrogen source is potassium nitrate.

10. The method of claim 9, wherein the nutshell is one of a coconut shell and a walnut shell.

11. The method of claim 7, wherein in step b) of conducting strain pouring, the biocarrier is one of sodium alginate, a mycelium pellet, and a polyurethane foam.

12. The method of claim 7, wherein in step a) of conducting strain screening, a concentration ratio of various substances in the screening medium is: 20 g/L of agar, 1 g/L of KNO₃, 1 g/L of KH₂PO₄, 0.5 g/L of FeCl₂.6H₂O, 0.2 g/L of CaCl₂.7H₂O, 1 g/L of MgSO₄.7H₂O, 8.5 g/L of sodium succinate, and 1 mL/L of bromothymol blue.

13. The method of claim 7, wherein in step c) of conducting strain screening, a concentration ratio of various substances in the liquid medium is: 1 g/L of KNO₃, 1 g/L of KH₂PO₄, 0.05 g/L of FeCl₂.6H₂O, 0.02 g/L of CaCl₂.7H₂O, 1 g/L of MgSO₄.7H₂O, and 8.5 g/L of sodium succinate, and a liquid medium is sterilized at 120° C. for 20-25 min, and cooled for later use.
